(19) United States
(12) Patent Application Publication
(10) Pub. No.: US 2024/0199529 A1
(43) Pub. Date: Jun. 20, 2024

(54) PROCESS FOR PREPARING ANILINE OR AN ANILINE DERIVATIVE

(71) Applicant: Covestro Deutschland AG, Leverkusen (DE)

(72) Inventors: Martin Machat, Köln (DE); Jens Langanke, Mechernich (DE); Christoph Guertler, Köln (DE); Marius Maronna, Aachen (DE); Tofighi Ghazal, Aachen (DE); Christine Hussong, Aachen (DE)

(21) Appl. No.: 18/565,264

(22) PCT Filed: Jun. 1, 2022
(86) PCT No.: PCT/EP2022/064887

§ 371 Date: Nov. 29, 2023

(30) Foreign Application Priority Data
Jun. 2, 2021 (EP) 21177307.2

(51) Int. Cl. C07C 209/78; B01J 21/04; C12P 13/00

(52) U.S. Cl. CPC C07C 209/78; B01J 21/04; C12P 13/001

(57) ABSTRACT

The present invention relates to a process for preparing aniline or an aniline derivative, comprising the steps of (I) providing aminobenzoic acid, (II) decarboxylating the aminobenzoic acid to aniline in the presence of an inorganic heterogeneous metal oxide catalyst containing, in relation to the total mass of metal oxides, a mass fraction of Al₂O₃ of 40.0% to 100%, preferably 50.0% to 100%, particularly preferably 60.0% to 100%, the mass fraction of Al₂O₃, in relation to the total mass of the inorganic heterogeneous metal oxide catalyst, being 25% to 100%, and (III) optionally reacting the aniline to form an aniline derivative.

The invention on which this application is based was financially supported by the German Federal Ministry of Food and Agriculture as part of the project “Biobasierte Herstellung von Intermediaten für Polyurethane—Phase II (Bio4PURPro)” [“Biobased Preparation of Intermediates for Polyurethanes—Phase II (Bio4PURPro)”] (funding code 22019918).

The present invention relates to a process for preparing aniline or an aniline conversion product, comprising the steps of (I) providing aminobenzoic acid, (II) decarboxylating the aminobenzoic acid to aniline in the presence of an inorganic heterogeneous metal oxide catalyst containing a proportion by mass of Al₂O₃ based on the total mass of the metal oxides of 40.0% to 100%, preferably 50.0% to 100%, particularly preferably 60.0% to 100%, the proportion by mass of Al₂O₃ based on the total mass of the inorganic heterogeneous metal oxide catalyst being 25% to 100%, and (III) optionally converting the aniline to an aniline conversion product.

The preparation of aniline by decarboxylation of aminobenzoic acid is known in principle in the prior art. By way of example, reference may be made to the international patent applications WO 2015/124686 A1 and WO 2015/124687 A1 and the literature cited therein. The aminobenzoic acid starting compound can be obtained chemically or preferably fermentatively.

The chemical preparation of aminobenzoic acid is known. A suitable synthesis route is, for example, the reaction of phthalimide with sodium hypochlorite. Phthalimide can itself be obtained from phthalic anhydride and ammonia.

The fermentative preparation of aminobenzoic acid is likewise known and described in the literature; see, for example, the applications WO 2015/124686 A1 and WO 2015/124687 A1 already mentioned and the literature cited in each.

WO 2015/124686 A1 describes the decarboxylation of fermentatively or chemically prepared anthranilic acid with extraction of aniline formed in the decarboxylation with an organic solvent extraneous to the system (an alcohol, phenol, amide, ether or aromatic hydrocarbon; in particular, 1-dodecanol is emphasized as a suitable solvent). Catalysts described for the decarboxylation are acidic catalysts such as zeolites or basic catalysts such as Mg—Al hydrotalcite (Mg₆Al₂(CO₃)(OH)₁₆·4H₂O, corresponding to a theoretical proportion by mass of “Al₂O₃” of 16.88%).

WO 2015/124687 A1 describes the performance of the decarboxylation of fermentatively prepared anthranilic acid in solvents including water or an organic solvent extraneous to the system, in particular 1-dodecanol, optionally in a mixture with aniline (cf. page 18, lines 28 and 29). In addition, this document also describes the option of performing the decarboxylation in aniline (without 1-dodecanol; see FIGS. 35 and 37 to 38 and the accompanying passages of text), optionally in the presence of 10% by mass of water (see FIG. 36 and the accompanying passages of text).

WO 2018/002088 A1 describes a process in which aminobenzoic acid is decarboxylated in a mixture with crude aniline. The crude aniline originates from the process itself, in that some of the product stream is not sent for purification but rather is recycled into the process. Catalysts described for the performance of the decarboxylation are aqueous acids such as sulfuric acid, nitric acid and hydrochloric acid; solid acids such as zeolites and Si—Ti molecular sieves, solid bases such as hydroxyapatite and hydrotalcite; and polymeric acids such as ion exchange resins (particularly Amberlyst).

WO 2020/020919 A1 describes the decarboxylation of aminobenzoic acid with exclusive use of the product aniline as catalyst. Catalysts extraneous to the system are deliberately avoided.

None of the processes described above is completely satisfactory in terms of economic viability, particularly with regard to the yield of aniline (suppression of the by-product 2-aminobenzanilide, which not only increases the proportion of aniline formed, but also facilitates the as quantitative as possible isolation thereof by means of distillative purification—as a result of reduced aniline losses via the necessary discharging of high boilers from the bottom). Further improvements in the preparation of aniline and aniline conversion products by decarboxylation of aminobenzoic acid, in particular fermentatively obtained aminobenzoic acid, would therefore be desirable.

Taking account of the above, the invention provides the following:

A process for preparing aniline or an aniline conversion product, comprising the steps of:
(I) providing aminobenzoic acid (in particular ortho-aminobenzoic acid);
(II) decarboxylating the aminobenzoic acid to aniline in the presence of an inorganic heterogeneous metal oxide catalyst (also referred to hereinafter as catalyst for short) containing a proportion by mass of Al₂O₃ based on the total mass of the metal oxides of 40.0% to 100%, preferably 50.0% to 100%, particularly preferably 60.0% to 100%, the proportion by mass of Al₂O₃ based on the total mass of the inorganic heterogeneous metal oxide catalyst being 25% to 100%; and
(III) optionally converting the aniline to an aniline conversion product.

In the terminology of the present invention, a metal oxide catalyst means a catalyst which contains at least one metal oxide or can be represented in terms of formula as containing at least one metal oxide. The total mass of the metal oxides refers to the maximum number of metal oxides which can be represented in terms of formula. If, for example, the composition of a catalyst can be represented in terms of formula as a “mixture” of aluminum oxide (Al₂O₃), magnesium oxide (MgO) and water (H₂O) (for instance “mAl₂O₃·nMgO·o H₂O”—first formula), then it is generally also possible to describe the same catalyst in a second formula as a “mixture” of aluminum hydroxide (Al(OH)₃) or aluminum oxide hydroxide (AlO(OH)) and magnesium hydroxide (Mg(OH)₂), with neither one nor the other formula necessarily being a correct representation of the actual structure. To determine the proportion by mass of Al₂O₃ for the purposes of the present invention, that description in terms of the formula which contains the maximum number of metal oxides is taken as a basis, regardless of whether or not the formula drawn up in this way adequately reflects the actual structure of the catalyst. In this sense, the proportion by mass of Al₂O₃ in the sense of the present invention can therefore be a theoretical value. The first formula is therefore decisive in the example chosen. The same applies to the proportions by mass of any further metal oxides.

What follows first is a brief summary of different possible embodiments of the invention, although the enumeration of embodiments should be considered to be nonexhaustive: In a first embodiment of the invention, which may be combined with all other embodiments, the inorganic heterogeneous metal oxide catalyst contains MgO in a proportion by mass based on the total mass of the metal oxides of 1.0% to 60.0%, preferably 2.0% to 50.0%, particularly preferably 5.0% to 35.0%.

In a second embodiment of the invention, which may be combined with all other embodiments, the inorganic heterogeneous metal oxide catalyst contains SiO₂ in a proportion by mass based on its total mass of 1.0% to 30.0%, preferably 2.0% to 20.0%, particularly preferably 2.0% to 10.0%.

In a third embodiment of the invention, which may be combined with all other embodiments, the Al₂O₃ comprises γ-Al₂O₃ or η-Al₂O₃, the catalyst preferably not comprising any further metal oxides.

In a fourth embodiment of the invention, which may be combined with all other embodiments, the decarboxylation of the aminobenzoic acid is performed at a temperature of 150° C. to 300° C., preferably 160° C. to 280° C., very particularly preferably 180° C. to 240° C.

In a fifth embodiment of the invention, which may be combined with all other embodiments, the decarboxylation of the aminobenzoic acid is performed at an absolute pressure of 0.05 bar to 300 bar, preferably 1.0 bar to 100 bar, particularly preferably 1.0 bar to 60 bar.

In a sixth embodiment of the invention, which may be combined with all other embodiments, the decarboxylation of the aminobenzoic acid is performed in the presence of aniline.

In a seventh embodiment of the invention, which is a particular configuration of the sixth embodiment and may otherwise be combined with all other embodiments as long as they do not provide for the decarboxylation to be performed continuously, the decarboxylation of the aminobenzoic acid is performed batchwise, with a proportion by mass of aniline, based on the total mass of aniline and aminobenzoic acid, of 0.1% to 90%, preferably 1.0% to 70%, particularly preferably of 5.0% to 50%, being established before the start of the decarboxylation.

In an eighth embodiment of the invention, which is a further particular configuration of the sixth embodiment and may otherwise be combined with all other embodiments as long as they do not provide for the decarboxylation to be performed batchwise, the decarboxylation of the aminobenzoic acid is performed continuously, with a proportion by mass of aniline, based on the total mass of aniline and aminobenzoic acid, of 0.1% to 90%, preferably 1.0% to 70%, particularly preferably 5.0% to 50%, always being established during the decarboxylation.

In a ninth embodiment of the invention, which may be combined with all other embodiments, the decarboxylation of the aminobenzoic acid is performed
in the liquid or gas phase in a reactor (in particular in a tubular reactor) with an integrated fixed bed of the inorganic heterogeneous metal oxide catalyst (containing a bed of the catalyst as shaped bodies (extrudates) or a configuration of the catalyst as a monolithic structure),
in the liquid or—preferably—gas phase in a fluidized bed reactor or
in the liquid phase in a stirred tank with a suspension (slurry) of the inorganic heterogeneous metal oxide catalyst contained therein.

In a tenth embodiment of the invention, which is a particular configuration of the ninth embodiment, the decarboxylation of the aminobenzoic acid is performed in the liquid or gas phase in a reactor (in particular in a tubular reactor) with an integrated fixed bed of the inorganic heterogeneous metal oxide catalyst containing a bed of the catalyst as shaped bodies (extrudates) or a configuration of the catalyst as a monolithic structure, the inorganic heterogeneous metal oxide catalyst being regenerated and reused after decarboxylation.

In an eleventh embodiment of the invention, which may be combined with all other embodiments, step (I) comprises the fermentation of a raw material containing a fermentable carbon-containing compound and a nitrogen-containing compound in the presence of microorganisms.

In a twelfth embodiment of the invention, which is a particular configuration of the eleventh embodiment, the fermentable carbon-containing compound comprises starch hydrolyzate, sugarcane juice, sugarbeet juice, hydrolyzates of lignocellulose-containing raw materials or a mixture of two or more of the aforementioned compounds, the nitrogen-containing compound comprising gaseous ammonia, aqueous ammonia, ammonium salts, urea or a mixture of two or more of the aforementioned compounds.

In a thirteenth embodiment of the invention, which is a particular configuration of the eleventh and twelfth embodiment, the microorganisms comprise Escherichia coli, Pseudomonas putida, Corynebacterium glutamicum, Ashbya gossypii, Pichia pastoris, Hansenula polymorpha, Yarrowia lipolytica, Zygosaccharomyces bailii or Saccharomyces cerevisiae.

In a fourteenth embodiment of the invention, which may be combined with all other embodiments, step (III) is performed and comprises one of the following conversions:
(I) acid-catalyzed reaction of the aniline with formaldehyde to form di- and polyamines of the diphenylmethane series;
(2) acid-catalyzed reaction of the aniline with formaldehyde to form di- and polyamines of the diphenylmethane series, followed by reaction thereof with phosgene to form di- and polyisocyanates of the diphenylmethane series; or
(3) conversion of the aniline to an azo compound.

In a fifteenth embodiment of the invention, which may be combined with all other embodiments, the aminobenzoic acid provided in step (A) comprises ortho-aminobenzoic acid (anthranilic acid) and in particular is ortho-aminobenzoic acid (i.e. does not comprise any further isomers of aminobenzoic acid).

The embodiments briefly outlined above and further possible configurations of the invention are elucidated in more detail hereinafter. In this context, all embodiments and further configurations of the invention are combinable as desired with one another unless the opposite is clearly apparent to a person skilled in the art from the context or any different statement is made explicitly.

The aminobenzoic acid to be provided in step (I) can be obtained in principle in any way known to experts. One option is the preparation of aminobenzoic acid by a chemical route. Preference is given to those processes that selectively afford the ortho isomer of aminobenzoic acid. One example of a suitable chemical method is the reaction of phthalimide with sodium hypochlorite. Phthalimide can itself be obtained from phthalic anhydride and ammonia.

Para-aminobenzoic acid can be prepared by a chemical route via the nitration of toluene with nitric acid, subsequent oxidation of the resulting para-nitrotoluene with oxygen to give para-nitrobenzoic acid and finally reduction with hydrazine to give para-aminobenzoic acid.

The preparation of meta-aminobenzoic acid is accomplished, for example, starting from methyl benzoate. Methyl meta-nitrobenzoate is obtained by nitrating methyl benzoate with nitric acid. This methyl ester is subsequently saponified with aqueous sodium hydroxide solution. Meta-nitrobenzoic acid is obtained after neutralization with hydrochloric acid and is finally reduced with hydrazine to give meta-aminobenzoic acid.

According to the invention, however, it is preferable to prepare the aminobenzoic acid required for the performance of step (I) by a fermentative process. In this embodiment of the invention, the provision of aminobenzoic acid comprises the fermentation of a raw material comprising at least a fermentable carbon-containing compound and a nitrogen-containing compound using microorganisms to obtain an aminobenzoate- and/or aminobenzoic acid-containing fermentation broth. This step may be performed by any fermentation process which is known from the prior art and is suitable for the preparation of aminobenzoic acid.

A fermentable carbon-containing compound in the context of this embodiment of the present invention is understood to mean any organic compound or mixture of organic compounds that can be used to produce aminobenzoic acid by the recombinant cells of the microorganism used. The production of aminobenzoic acid can take place here in the presence or in the absence of oxygen. Preference is given here to those fermentable carbon-containing compounds which can additionally serve as energy and carbon source for the growth of the recombinant cells of the microorganism used. Particularly suitable are starch hydrolyzate, sugarcane juice, sugarbeet juice and hydrolyzates of lignocellulose-containing raw materials, and mixtures thereof (i.e. mixtures of two or more of the aforementioned compounds). Likewise suitable are glycerol and C1 compounds, particularly carbon monoxide. Useful nitrogen-containing compounds suitable for step (I)(1) include in particular gaseous ammonia, aqueous ammonia, ammonium salts (in particular inorganic ammonium salts such as ammonium chloride and/or ammonium sulfate, preferably ammonium sulfate), urea or mixtures thereof (i.e. mixtures of two or more of the aforementioned compounds).

Preferred microorganisms for the performance of the fermentation are bacteria or fungi, especially yeasts. Particularly preferred here are microorganisms such as Escherichia coli, Pseudomonas putida, Corynebacterium glutamicum, Ashbya gossypii, Pichia pastoris, Hansenula polymorpha, Kluyveromyces marxianus, Yarrowia lipolytica, Zygosaccharomyces bailii or Saccharomyces cerevisiae, particular preference being given to the sole use of Corynebacterium glutamicum, in particular Corynebacterium glutamicum ATCC 13032. The pH to be maintained in the fermentation is guided by the microorganism used. Microorganisms such as Corynebacterium glutamicum, Pseudomonas putida or Escherichia coli are preferably cultured at neutral pH (i.e. at a pH in the range from 6.0 to 8.0). Microorganisms such as Saccharomyces cerevisiae, by contrast, are preferably cultured in acidic medium (i.e. at a pH in the range from 3.0 to 6.0).

In any case, the microorganism of the fermentation is preferably selected such that the ortho isomer of aminobenzoic acid is formed in the fermentation.

In a preferred configuration of the invention, bacteria are used as microorganisms. In this connection, reference is made in particular to patent applications WO 2015/124686 A1 and WO 2015/124687 A1, which describe a fermentation usable according to the invention with use of bacteria (see, for example, WO 2015/124687 A1, (i) page 15, line 8 to page 16, line 30, (ii) example 1 (page 29, lines 4 to 26), (iii) example 3 (especially page 34, lines 10 to 18), (iv) example 4 (especially page 55, lines 9 to 31). In particular, use is made of bacteria which are capable of converting a fermentable carbon-containing compound to aminobenzoic acid in the presence of a suitable nitrogen source without the aminobenzoic acid thus formed being consumed straight away in intracellular biochemical processes, with the result that aminobenzoic acid is enriched in the cell and is ultimately transferred into the fermentation broth.

In another preferred configuration of the invention, yeasts are used as microorganisms. In this connection, reference is made in particular to international patent application WO 2017/102853 A1. In particular, use is made of yeast cells which are capable of converting a fermentable carbon-containing compound to aminobenzoic acid in the presence of a suitable nitrogen source without the aminobenzoic acid thus formed being consumed straight away in intracellular biochemical processes, with the result that aminobenzoic acid is enriched in the cell and is ultimately transferred into the fermentation broth.

Suitable bacteria or yeast cells can be identified, for example, by screening for mutants which secrete aminobenzoic acid into the surrounding medium. However, preference is given to the specific modification of key enzymes by means of genetic engineering methods. Using customary genetic engineering methods, gene expression and enzyme activity can be enhanced, reduced or even completely suppressed as desired. Recombinant strains are the result.

In the majority of cases, the fermentation broth present at the end of the fermentation is basic to neutral or slightly acidic at most (pH>4.7), and the aminobenzoic acid is consequently in the form of its aminobenzoate anion. In these cases, it is preferable to treat the fermentation broth with acid, especially with hydrochloric acid, sulfuric acid and/or phosphoric acid, in order to convert the anion to the electronically uncharged form. The acid is added in particular until the pH of the resulting mixture is in the range from 3.0 to 4.7, preferably in the range from 3.2 to 3.7 (in particular in the case of meta- and para-aminobenzoic acid), particularly preferably in the range from 3.4 to 3.6 (in particular in the case of ortho-aminobenzoic acid). Aminobenzoic acid is then predominantly to completely in the electronically uncharged form and, owing to the low water solubility thereof, precipitates out apart from a small proportion attributable to a certain residual solubility, and can easily be separated from the supernatant fermentation broth, in particular by filtration or centrifugation. Filtration can be performed at reduced pressure, atmospheric pressure or elevated pressure. Centrifugation can be performed using commercial centrifuges. It is also possible to leave the suspension obtained in the acid treatment standing until the precipitated crystals of aminobenzoic acid settle out and to then decant the supernatant mother liquor or filter it off with suction.

Should the fermentation broth however be strongly acidic (pH<3.0), a pH in the aforementioned ranges is ensured by adding base (preferably aqueous sodium hydroxide solution, lime). If the pH of the fermentation broth, by contrast, is in the range from 3.0 to 4.0, as can be the case when using yeasts as microorganisms, neither acid nor base is added in a preferred embodiment, but rather the fermentation broth is processed further directly without further pH adjustment. In this case, it is to be expected that crystals of aminobenzoic acid will spontaneously precipitate and can be directly separated off. With regard to the methods employable for this purpose, the statements made above for the acid treatment are applicable.

Any necessary separation of solid aminobenzoic acid and solid microorganisms present in aqueous solution is best accomplished through centrifugation. This is true of all embodiments of the present invention in which such a separation is required.

The aminobenzoic acid obtained in one of the ways described above may be processed further prior to the performance of the decarboxylation. Preference is given to scrubbing with aqueous wash media, in particular water. In order to avoid yield losses, the pH of the aqueous wash medium can be adjusted to the same pH as after the end of addition of acid (or in the case of yeasts: addition of base); thus, in this embodiment, washing is performed with a dilute acid rather than with water. Suitable acids for this purpose are the acids mentioned above in connection with the acid treatment.

The aminobenzoic acid thus provided chemically or fermentatively is decarboxylated to aniline in step (II). According to the invention, the catalyst used for this purpose features a high proportion by mass of aluminum oxide (at least 40%, determined as described further above). The aluminum oxide is preferably γ-Al₂O₃ or η-Al₂O₃, especially when no further metal oxides are present in addition to aluminum oxide. In addition to aluminum oxide, further metal oxides can in principle also be present, in particular contains magnesium oxide (MgO) in a proportion by mass based on the total mass of the metal oxides of 1.0% to 60.0%, preferably 2.0% to 50.0%, particularly preferably 5.0% to 35.0%. Furthermore, the catalyst to be used according to the invention may contain SiO₂ in a proportion by mass based on its total mass of 1.0% to 30.0%, preferably 2.0% to 20.0%, particularly preferably 2.0% to 10.0%.

With regard to the reaction conditions, the decarboxylation may thus be performed within a wide range of temperature and pressure. A suitable reaction temperature is preferably a temperature in the range from 150° C. to 300° C., particularly preferably 160° C. to 280° C., very particularly preferably 180° C. to 240° C. The (absolute) reaction pressure here may be 0.05 bar to 300 bar, preferably 1.0 bar to 100 bar, particularly preferably 1.0 bar to 60 bar.

In one embodiment of the invention, the decarboxylation is effected in the presence of aniline, i.e. the aminobenzoic acid is dissolved in aniline. In the case of batchwise performance of the reaction, a proportion by mass of aniline, based on the total mass of aniline and aminobenzoic acid, of 0.1% to 90%, preferably 1.0% to 70%, particularly preferably of 5.0% to 50%, is preferably established before the start of the decarboxylation. In the case of continuous performance of the reaction, a proportion by mass of aniline, based on the total mass of aniline and aminobenzoic acid, of 0.1% to 90%, preferably 1.0% to 70%, particularly preferably 5.0% to 50%, is always established during the decarboxylation.

Other solvents or diluents may of course also be used in addition to aniline, in particular water. Further suitable are preferably organic, polar or protic solvents such as halogenated aliphatic or aromatic hydrocarbons, linear or cyclic ethers, linear or cyclic esters, linear or cyclic amides, alcohols, ketones, nitriles, phenol derivatives, benzanilides, sulfonamides or sulfolane, preferably having a boiling point which, under the chosen conditions, is higher than the chosen reaction temperature and at this temperature preferably forms a homogeneous reaction mixture with the reaction components.

With regard to the reaction regime, both performance in the gas phase and in the liquid phase are suitable. The reaction may be performed continuously (preferably) or batchwise.

Preferred procedures comprise the performance of the decarboxylation of the aminobenzoic acid
in the liquid or gas phase in a reactor, in particular in a tubular reactor, with an integrated fixed bed of the catalyst (containing a bed of the catalyst as shaped bodies (extrudates) or a configuration of the catalyst as a monolithic structure),
in the liquid or—preferably—gas phase in a fluidized bed reactor or
in the liquid phase in a stirred tank with a suspension (slurry) of the catalyst contained therein.

In the context of the present invention, a tubular reactor is understood to mean a tube-shaped reactor through which, in the case of a continuous reaction regime (which is preferred), the reacting reaction mixture flows during operation. Tubular reactors with small ratios of length to diameter are also referred to as tower reactors; these are also included in the understanding according to the invention of the term tubular reactor.

The use of shaped catalyst bodies (extrudates) or monolithic catalyst structures allows the catalyst to be easily reused after decarboxylation. In the case of a batchwise process regime, “after decarboxylation” means after reaching the maximum conversion of a batch of aminobenzoic acid to be converted. In the case of a continuous process regime, “after decarboxylation” means the point in time at which the conversion drops to significantly below the initial conversion (the conversion at the beginning of a new reaction cycle with a new or regenerated catalyst) (in particular drops to a value of 97.0% or less of the initial conversion) and the reaction is therefore no longer continued. All methods known to experts for monitoring continuous reactions are suitable for determining the conversion, in particular high-performance liquid chromatography (HPLC) or gas chromatography (GC). The standard methods generally deliver consistent results within the accuracy of measurement. If, contrary to expectations, various methods should yield significantly different results for the conversion at a specific point in time, then determining the conversion by way of high-performance liquid chromatography (HPLC) is decisive for the purposes of the present invention.

The catalyst present after decarboxylation is regenerated before it is used again. For this purpose, the catalyst may be washed with organic solvent or aqueous solutions and/or burnt out at elevated temperature in the presence of O₂ in order to remove organic deposits.

The aniline formed may be isolated and purified using standard techniques, in particular by distillation.

The aniline obtained in this way may be supplied to a wide variety of subsequent applications in step (III) to form an aniline conversion product. Examples include the following conversions:
(1) acid-catalyzed reaction of the aniline with formaldehyde to form di- and polyamines of the diphenylmethane series;
(2) acid-catalyzed reaction of the aniline with formaldehyde to form di- and polyamines of the diphenylmethane series, followed by reaction thereof with phosgene to form di- and polyisocyanates of the diphenylmethane series; or
(3) conversion of the aniline to an azo compound.

The further reaction of aniline with formaldehyde to give di- and polyamines of the diphenylmethane series (III)(1) is known per se and may be performed by any prior art process. The continuous or partially batchwise preparation of di- and polyamines of the diphenylmethane series from aniline and formaldehyde is disclosed, for example, in EP 1 616 890 A1, U.S. Pat. No. 5,286,760, EP-A-451442 and WO-A-99/40059. The reaction is effected under acid catalysis. A suitable acidic catalyst is preferably hydrochloric acid.

The further reaction of the di- and polyamines of the diphenylmethane series that are thus obtained with phosgene to give di- and polyisocyanates of the diphenylmethane series (III)(2) is also known per se and may be performed by any prior art process. Suitable processes are described, for example, in EP 2 077 150 B1, EP 1 616 857 A1, EP 1 873 142 A1, and EP0 314 985B1.

The conversion of the aniline obtained according to the invention to azo compounds, in particular to azo dyes (III)(3) may also be effected by any prior art process. By way of example, reference may be made to the known preparation of Aniline Yellow (para-aminoazobenzene; CAS 493-5-7) or Indigo (2,2′-bis(2,3-dihydro-3-oxomethylidene); CAS 482-89-3).

The invention is elucidated in more detail with reference to the examples which follow.

### EXAMPLES

### Compounds Used:

### Reactants:

Anthranilic acid (AA, petrochemical): C₇H₇NO₂, purity ≥98%, Acros Organics
Anthranilic acid (AA, biogenic): C₇H₇NO₂, purity: 98%, Covestro Deutschland AG
Aniline (ANL): C₆H₇N, purity ≥99.5%, Sigma-Aldrich
2-Aminobenzanilide (AMD): C₁₃H₁₂N₂O, purity 95%, abcr GmbH
Demineralized water: deionized

### Catalysts:

TiO₂ (Anatas, ST61120), Saint-Gobain
SiO₂ (SS61138), Saint-Gobain
Mesostructured SiO₂ (MCM-41, ˜1000 m²/g BET-SA, Sigma-Aldrich)
ZrO₂ (tetragonal, SZ61152), Saint-Gobain
W-doped ZrO₂ (SZ61143), Saint-Gobain
ZnO (≥99%), Sigma-Aldrich
Pural Zn44 (44% Zn in Al₂O₃), Sasol
MgO (purity ˜98%), Acros Organics
Pural MG5 (mixed oxide, MgO:Al₂O₃ mass ratio of 5:95), Sasol
Pural MG20 (MgO:Al₂O₃ mass ratio of 20:80), Sasol
Pural MG30 (spinel-type structure, MgO:Al₂O₃ mass ratio of 30:70), Sasol
Al₂O₃ (AI 4126 E), BASF
γ-Al₂O₃(SA6175), Saint-Gobain
Silylated γ-Al₂O₃(SA6175), Saint-Gobain
Pural TH100-AlO(OH), Sasol
η-Al₂O₃(Eta-Alox V1900)
Siralox 5 (SiO₂:Al₂O₃ mass ratio of 5:95), Sasol
Siralox10 (SiO₂:Al₂O₃ mass ratio of 10:90), Sasol
Siralox30 (SiO₂:Al₂O₃ mass ratio of 30:70), Sasol
Zeolite CBV600 (Na₂O·Al₂O₃·SiO₂, SiO₂/Al₂O₃ mass ratio=5.2; containing 0.2% by mass of Na₂O), Zeolyst International.
Puralox SCFa-160/Ce20, 160 m²/g BET-SA, Sasol
Puralox SCFa-190/Zr20, 190 m²/g BET-SA, Sasol
Puralox TH100-150/Ti10, 150 m²/g BET-SA, Sasol
Puralox TH100/150/L4, 150 m²/g BET-SA, Sasol

### Catalyst Preparation:

All catalysts for reactions under slurry conditions were sieved (45-90 μm) and dried for 3 h at 10 mbar and 200° C. before they were used. The catalysts were then stored under an Ar atmosphere until they were used.

### Method Description:

HPLC: For HPLC analysis, a setup from Agilent with UV detection (DAD, measured at 254 nm) was used. For separation, a column from Agilent (Eclipse XDB-C18; 5 μm; 4.6×150 mm) was used. The flow agent used was a mixture of MeOH and buffer (MeOH:buffer volume ratio=40:60, buffer: 0.7 ml of 85% p.a. H₃PO₄ is diluted to a final volume of 1 I with HPLC water, where the pH of 3.0 is to be set with aqueous sodium hydroxide solution before final filling). The flow rate was 0.7 ml/min. The temperature of the column oven was adjusted to 40° C. The injection volume was 1 μl. The retention times of the individual components aniline (ANL), anthranilic acid (AA) and 2-aminobenzanilide (ABD) were: ANL=3.2 min; AA=5.2 min; amide=15.7 min.

The peak areas are converted to area percent (A %). The quantification of the individual components in percent by mass (wt. %), based on the reaction mixture, was enabled by calibration with pure substances beforehand. In addition to the mass composition, the values are used to determine the conversion of anthranilic acid, the yield of aniline formed, the selectivity of the aniline formation and the selectivity for the formation of 2-aminobenzanilide.

### General Procedure 1: Decarboxylation of Anthranilic Acid (Slurry; Ex. 1 to 62)

The decarboxylation of anthranilic acid is performed in a steel reactor charged with 1.6 g of anthranilic acid, aniline (optional, see Table 1 for amount), pulverulent catalyst (optional, see Table 2 for amount) and demineralized water (optional, see Table 1 for amount). The steel reactor is then closed and purged with Ar. The reaction mixture is then stirred for a defined reaction time at a defined temperature and 360 rpm. This increasingly builds up pressure due to the release of CO₂. Subsequently, the reaction mixture is cooled in an ice bath, the pressure is released and the mixture is diluted with 4.0 g of methanol. The diluted mixture is filtered and characterized by means of HPLC analysis.

### General Procedure 2: Decarboxylation of Anthranilic Acid (Extrudates; Ex. 63 to 74)

To decarboxylate anthranilic acid with extrudates, Al₂O₃ extrudates (BASF; AI 4126 E) in cylindrical form (cross section diameter=3 mm, length≈4.5 mm) are used and the recyclability over 6 catalyst recycling cycles is investigated. For this purpose, 1.6 g of anthranilic acid is added into an autoclave with a stainless steel cage containing 0.2 g of Al₂O₃ extrudates. 0.25 g of distilled water (13.5% by weight) is then added. After reducing the pressure to 100 mbar and purging the reactor with Ar gas, the reaction mixture is heated to 225° C. and stirred for 1 h (360 rpm). This increasingly builds up pressure due to the release of CO₂. Subsequently, the reaction mixture is cooled in an ice bath, the pressure is released and the mixture is diluted with approx. 4.0 g of methanol. The diluted mixture is characterized by means of HPLC analysis. The used extrudates are then used for the next reaction cycle.

The experiments are summarized in the tables below. The following abbreviations are used therein:
C=Comparative example
Cat.=Catalyst
ANL=Aniline
AA=Anthranilic acid
AMD=2-Aminobenzanilide

TABLE 1
Metal oxide compositions of the tested catalysts

| | | Catalyst | | | | | | | |
|---|---|---|---|---|---|---|---|---|---|
| | | Al₂O₃ | MgO | SiO₂ | TiO₂ | ZnO | ZrO₂ | CeO₂ | La₂O₃ |
| | | Proportion by mass in %, based on the total mass of all metal | | | | | | | |
| No. | Name | oxides present in the material | | | | | | | |
| K1 | TiO₂ (Anatas) | 0 | 0 | 0 | 100 | 0 | 0 | 0 | 0 |
| K2 | SiO₂ | 0 | 0 | 100 | 0 | 0 | 0 | 0 | 0 |
| K3 | MCM41/SiO₂ | 0 | 0 | 100 | 0 | 0 | 0 | 0 | 0 |
| K4 | ZrO₂ | 0 | 0 | 0 | 0 | 0 | 100 | 0 | 0 |
| K5 | W-doped ZrO₂ | 0 | 0 | 0 | 0 | 0 | ≥90 | 0 | 0 |
| K6 | ZnO | 0 | 0 | 0 | 0 | 100 | 0 | 0 | 0 |
| K7 | MgO | 0 | 100 | 0 | 0 | 0 | 0 | 0 | 0 |
| K8 | Zeolite CBV 600 | 24.6 | 0 | 75.4 | 0 | 0 | 0 | 0 | 0 |
| K9 | η-Al₂O₃ | 100 | 0 | 0 | 0 | 0 | 0 | 0 | 0 |
| K10 | γ-Al₂O₃ | 100 | 0 | 0 | 0 | 0 | 0 | 0 | 0 |
| K11 | γ-Al₂O₃; silylated | 100 | 0 | 0 | 0 | 0 | 0 | 0 | 0 |
| K12 | Al₂O₃ (BASF, Al | 100 | 0 | 0 | 0 | 0 | 0 | 0 | 0 |
| | 4126 E) | | | | | | | | |
| K13 | TH100-AIO(OH) | 100 | 0 | 0 | 0 | 0 | 0 | 0 | 0 |
| | (boehmite) | | | | | | | | |
| K14 | Pural ® ZN44 | 56 | 0 | 0 | 0 | 44 | 0 | 0 | 0 |
| K15 | Pural ® MG5 | 95 | 5 | 0 | 0 | 0 | 0 | 0 | 0 |
| K16 | Pural ® MG20 | 80 | 20 | 0 | 0 | 0 | 0 | 0 | 0 |
| K17 | Pural ® MG30 | 70 | 30 | 0 | 0 | 0 | 0 | 0 | 0 |
| K19 | Siralox ® 5 | 95 | 0 | 5 | 0 | 0 | 0 | 0 | 0 |
| K20 | Siralox ® 10 | 90 | 0 | 10 | 0 | 0 | 0 | 0 | 0 |
| K21 | Siralox ® 30 | 70 | 0 | 30 | 0 | 0 | 0 | 0 | 0 |
| K22 | Puralox ® SCFa- | 79.7 | 0 | 0 | 0 | 0 | 0 | 20.3 | 0 |
| | 160/Ce20 | | | | | | | | |
| K23 | Puralox ® SCFa- | 79.6 | 0 | 0 | 0 | 0 | 20.4 | 0 | 0 |
| | 190/Zr20 | | | | | | | | |
| K24 | Puralox ®TH100- | 89.6 | 0 | 0 | 10.4 | 0 | 0 | 0 | 0 |
| | 150/Ti10 | | | | | | | | |
| K25 | Puralox ® | 96.0 | 0 | 0 | 0 | 0 | 0 | 0 | 4.0 |
| | TH100/150/L4 | | | | | | | | |

TABLE 2
Decarboxylation of anthranilic acid to aniline. Reaction temperature T = 185° C.; reaction
time t_{R} = 20 min. Varying catalysts.

| | | | AAₜ₌ₜ₀ | ANLₜ₌ₜ₀ | H₂O | ANL_{t=tR} | AA_{t=tR} | AMD_{t=tR} | A_{ANL} | X_{AA} | S_{ANL} | S_{AMD} |
|---|---|---|---|---|---|---|---|---|---|---|---|---|
| Ex. | Cat. [a] | | [b] | [b] | [b] | [c] | [c] | [c] | [d] | [e] | [f] | [f] |
| 1 (C) | — | 0 | 50.0 | 50.0 | 0 | 57.5 | 41.7 | 0.8 | 17.1 | 19.0 | 90.0 | 5.0 |
| 2 (C) | K1 | 5.9 | 50.0 | 50.0 | 0 | 58.1 | 41.0 | 0.9 | 18.2 | 20.4 | 89.4 | 5.3 |
| 3 (C) | K2 | 5.9 | 50.0 | 50.0 | 0 | 59.4 | 39.6 | 1.0 | 21.3 | 23.7 | 89.7 | 5.1 |
| 4 (C) | K3 | 5.9 | 50.0 | 50.0 | 0 | 61.4 | 37.5 | 1.1 | 25.3 | 28.0 | 90.4 | 4.8 |
| 5 (C) | K4 | 5.9 | 50.0 | 50.0 | 0 | 59.8 | 39.3 | 0.8 | 21.8 | 23.9 | 91.4 | 4.3 |
| 6 (C) | K5 | 5.9 | 50.0 | 50.0 | 0 | 65.1 | 34.1 | 0.8 | 32.9 | 34.9 | 94.1 | 3.0 |
| 7 (C) | K6 | 5.9 | 50.0 | 50.0 | 0 | 67.2 | 32.3 | 0.4 | 38.1 | 39.2 | 97.2 | 1.4 |
| 8 (C) | K7 | 5.9 | 50.0 | 50.0 | 0 | 70.3 | 29.2 | 0.6 | 44.4 | 45.8 | 97.1 | 1.5 |
| 9 (C) | K8 | 5.9 | 50.0 | 50.0 | 0 | 71.2 | 28.1 | 0.7 | 46.2 | 47.8 | 96.6 | 1.7 |
| 10 | K9 | 5.9 | 50.0 | 50.0 | 0 | 94.7 | 5.0 | 0.3 | 90.8 | 91.5 | 99.2 | 0.4 |
| 11 | K10 | 5.9 | 50.0 | 50.0 | 0 | 94.3 | 5.2 | 0.5 | 90.0 | 91.1 | 98.8 | 0.6 |
| 12 | K11 | 5.9 | 50.0 | 50.0 | 0 | 95.5 | 4.1 | 0.4 | 92.1 | 93.0 | 99.1 | 0.5 |
| 13 | K12 | 5.9 | 50.0 | 50.0 | 0 | 88.4 | 11.1 | 0.5 | 79.5 | 80.5 | 98.6 | 0.7 |
| 13a | K12 | 5.9 | 50.0 | 50.0 | 0 | 92.5 | 7.2 | 0.3 | 86.8 | 87.5 | 99.2 | 0.4 |
| | | | [g] | | | | | | | | | |
| 14 | K13 | 5.9 | 50.0 | 50.0 | 0 | 89.0 | 10.4 | 0.6 | 80.5 | 81.9 | 98.3 | 0.9 |
| 15 | K14 | 5.9 | 50.0 | 50.0 | 0 | 73.7 | 25.8 | 0.6 | 51.2 | 52.6 | 97.4 | 1.3 |
| 16 | K15 | 5.9 | 50.0 | 50.0 | 0 | 95.6 | 4.2 | 0.1 | 92.5 | 92.8 | 99.7 | 0.2 |
| 17 | K16 | 5.9 | 50.0 | 50.0 | 0 | 96.8 | 3.1 | 0.1 | 94.6 | 95.0 | 99.8 | 0.1 |
| 18 | K17 | 5.9 | 50.0 | 50.0 | 0 | 96.5 | 3.4 | 0.1 | 94.0 | 94.2 | 99.7 | 0.1 |
| 18a | K17 | 5.9 | 50.0 | 50.0 | 0 | 97.0 | 2.9 | 0.1 | 94.9 | 95.1 | 99.8 | 0.1 |
| | | | [g] | | | | | | | | | |
| 20 | K19 | 5.9 | 50.0 | 50.0 | 0 | 95.7 | 4.0 | 0.3 | 92.5 | 93.1 | 99.4 | 0.3 |
| 21 | K20 | 5.9 | 50.0 | 50.0 | 0 | 95.4 | 4.3 | 0.3 | 92.0 | 92.7 | 99.3 | 0.4 |
| 22 | K21 | 5.9 | 50.0 | 50.0 | 0 | 77.8 | 21.4 | 0.9 | 59.3 | 61.3 | 96.8 | 1.6 |
| 23 | K22 | 5.9 | 50.0 | 50.0 | 0 | 82.6 | 16.9 | 0.6 | 68.7 | 70.0 | 98.1 | 1.0 |
| 24 | K23 | 5.9 | 50.0 | 50.0 | 0 | 74.0 | 25.3 | 0.7 | 51.6 | 53.3 | 96.8 | 1.6 |
| 25 | K24 | 5.9 | 50.0 | 50.0 | 0 | 76.2 | 23.2 | 0.6 | 56.4 | 57.9 | 97.5 | 1.3 |
| 26 | K25 | 5.9 | 50.0 | 50.0 | 0 | 74.5 | 24.9 | 0.6 | 52.9 | 54.3 | 97.4 | 1.3 |

TABLE 3
Decarboxylation of anthranilic acid to aniline. Reaction temperature T = 185° C. Varying
reactant mixture compositions.

| | | | AAₜ₌ₜ₀ | ANLₜ₌ₜ₀ | H₂O | t_{R}/ | ANL_{t=tR} | AA_{t=tR} | AMD_{t=tR} | A_{ANL} | X_{AA} | S_{ANL} | S_{AMD} |
|---|---|---|---|---|---|---|---|---|---|---|---|---|---|
| Ex. | Cat. [a] | | [b] | [b] | [b] | min | [c] | [c] | [c] | [d] | [e] | [f] | [f] |
| 27 | — | 0 | 50.0 | 50.0 | 0 | 60 | 75.2 | 22.8 | 1.9 | 53.9 | 58.5 | 92.2 | 3.9 |
| 28 | K17 | 5.9 | 50.0 | 50.0 | 0 | 60 | 98.4 | 1.5 | 0.1 | 97.2 | 97.4 | 99.7 | 0.1 |
| 29 | — | 0 | 60.0 | 40.0 | 0 | 60 | 75.0 | 23.0 | 2.2 | 61.8 | 66.0 | 94.0 | 3.1 |
| (C) | | | | | | | | | | | | | |
| 30 | K17 | 6.9 | 60.0 | 40.0 | 0 | 60 | 98.2 | 1.7 | 0.1 | 97.5 | 97.7 | 99.7 | 0.1 |
| 31 | — | 0 | 72.0 | 28.0 | 0 | 60 | 72.8 | 24.7 | 2.5 | 67.7 | 71.4 | 94.8 | 2.6 |
| (C) | | | | | | | | | | | | | |
| 32 | K17 | 8.3 | 72.0 | 28.0 | 0 | 60 | 97.1 | 2.7 | 0.2 | 96.9 | 97.1 | 99.7 | 0.1 |
| 33 | — | 0 | 88.0 | 12.0 | 0 | 60 | 70.4 | 26.9 | 2.7 | 72.9 | 76.0 | 95.9 | 2.1 |
| (C) | | | | | | | | | | | | | |
| 34 | K17 | 10 | 88.0 | 12.0 | 0 | 60 | 91.0 | 8.6 | 0.4 | 92.5 | 92.9 | 99.5 | 0.2 |
| 35 | — | 0 | 100 | 0 | 0 | 60 | 70.0 | 27.1 | 3.0 | 77.0 | 80.0 | 96.4 | 1.8 |
| (C) | | | | | | | | | | | | | |
| 36 | K17 | 11 | 100 | 0 | 0 | 60 | 85.2 | 14.0 | 0.8 | 89.3 | 90.0 | 99.2 | 0.4 |
| 37 | K12 | 20 | 100 | 0 | 0 | 180 | 98.1 | 1.0 | 0.9 | 98.5 | 99.3 | 99.2 | 0.4 |
| 38 | K12 | 5.9 | 100 | 0 | 0 | 180 | 97.2 | 0.6 | 2.2 | 97.6 | 99.6 | 98.0 | 1.0 |

TABLE 4
Decarboxylation of anthranilic acid to aniline. Reaction temperature T = 185° C.; reaction
time t_{R} = 20 min. Varying water contents.

| | | | AAₜ₌ₜ₀ | ANLₜ₌ₜ₀ | H₂O | ANL_{t=tR} | AA_{t=tR} | AMD_{t=tR} | A_{ANL} | X_{AA} | S_{ANL} | S_{AMD} |
|---|---|---|---|---|---|---|---|---|---|---|---|---|
| Ex. | Cat. [a] | | [b] | [b] | [b] | [c] | [c] | [c] | [d] | [e] | [f] | [f] |
| 39 (C) | — | 0 | 48.0 | 48.0 | 5.0 | 58.6 | 40.7 | 0.7 | 18.9 | 20.7 | 91.2 | 4.4 |
| 40 (C) | — | 0 | 45.0 | 45.0 | 10.0 | 61.1 | 38.3 | 0.6 | 25.1 | 26.5 | 94.6 | 2.7 |
| 41 (C) | — | 0 | 43.0 | 43.0 | 13.0 | 63.4 | 36.2 | 0.4 | 30.1 | 31.1 | 96.6 | 1.7 |
| 42 | K12 | 5.6 | 48.0 | 48.0 | 5.0 | 94.5 | 5.3 | 0.2 | 90.5 | 90.9 | 99.5 | 0.2 |
| 43 | K12 | 5.4 | 45.0 | 45.0 | 10.0 | 94.0 | 5.9 | 0.1 | 89.5 | 89.8 | 99.6 | 0.2 |
| 44 | K12 | 5.2 | 43.0 | 43.0 | 13.0 | 94.8 | 5.1 | 0.1 | 90.9 | 91.2 | 99.7 | 0.1 |
| 45 | K10 | 5.6 | 48.0 | 48.0 | 5.0 | 94.5 | 5.4 | 0.1 | 90.4 | 90.7 | 99.7 | 0.2 |
| 46 | K10 | 5.4 | 45.0 | 45.0 | 10.0 | 95.3 | 4.7 | 0.1 | 91.8 | 92.0 | 99.8 | 0.1 |
| 47 | K10 | 5.2 | 43.0 | 43.0 | 13.0 | 95.6 | 4.3 | 0.1 | 92.5 | 92.7 | 99.8 | 0.1 |
| 48 | K17 | 5.6 | 48.0 | 48.0 | 5.0 | 96.7 | 3.2 | 0.05 | 94.4 | 94.6 | 99.9 | 0.06 |
| 49 | K17 | 5.4 | 45.0 | 45.0 | 10.0 | 96.0 | 4.0 | 0.04 | 93.1 | 93.2 | 99.9 | 0.05 |
| 50 | K17 | 5.2 | 43 | 43 | 13 | 93.6 | 6.4 | 0.02 | 89.0 | 89.1 | 99.9 | 0.03 |

TABLE 5
Decarboxylation of anthranilic acid to aniline. Varying water contents, reaction
temperatures and times, and different anthranilic acid sources with K17 and K12.

| | | | AAₜ₌ₜ₀ | ANLₜ₌ₜ₀ | H₂O | T/ | t_{R}/ | ANL_{t=tR} | AA_{t=tR} | AMD_{t=tR} | A_{ANL} | X_{AA} | S_{ANL} | S_{AMD} |
|---|---|---|---|---|---|---|---|---|---|---|---|---|---|---|
| Ex. | Cat. [a] | | [b] | [b] | [b] | ° C. | min | [c] | [c] | [c] | [d] | [e] | [f] | [f] |
| 51 | K17 | 5.9 | 50 | 50 | 0 | 185 | 60 | 98.9 | 1.0 | 0.1 | 98.1 | 98.3 | 99.8 | 0.1 |
| 52 | K17 | 5.6 | 48 | 48 | 5.0 | 185 | 60 | 98.9 | 1.1 | 0.04 | 98.1 | 98.2 | 99.9 | 0.05 |
| 53 | K17 | 5.9 | 50 | 50 | 0 | 200 | 20 | 98.7 | 1.2 | 0.1 | 97.8 | 97.9 | 99.8 | 0.1 |
| 54 | K17 | 5.9 | 50 | 50 | 0 | 200 | 60 | 99.1 | 0.8 | 0.1 | 98.4 | 98.7 | 99.8 | 0.1 |
| 55 | K17 | 5.6 | 48 | 48 | 5.0 | 200 | 60 | 99.3 | 0.6 | 0.1 | 98.8 | 98.9 | 99.9 | 0.06 |
| 56 | K17 | 5.9 | 50 | 50 | 0 | 225 | 20 | 98.7 | 1.2 | 0.1 | 97.8 | 98.0 | 99.8 | 0.1 |
| 57 | K17 | 5.9 | 50 | 50 | 0 | 225 | 60 | 99.2 | 0.7 | 0.1 | 98.6 | 98.9 | 99.7 | 0.1 |
| 57a | K17 | 5.9 | 50 [g] | 50 | 0 | 225 | 60 | 97.9 | 1.9 | 0.2 | 96.4 | 96.8 | 99.6 | 0.2 |
| 58 | K17 | 5.6 | 48 | 48 | 5.0 | 225 | 60 | 99.2 | 0.7 | 0.1 | 98.6 | 98.8 | 99.8 | 0.1 |
| 59 | K17 | 5.9 | 50 | 50 | 0 | 230 | 20 | 98.8 | 1.1 | 0.1 | 98.0 | 98.2 | 99.8 | 0.1 |
| 60 | K17 | 5.9 | 50 | 50 | 0 | 230 | 60 | 99.2 | 0.7 | 0.1 | 98.6 | 98.9 | 99.7 | 0.1 |
| 61 | K17 | 5.6 | 48 | 48 | 5.0 | 230 | 60 | 99.0 | 0.9 | 0.1 | 98.4 | 98.5 | 99.8 | 0.1 |
| 62 | K12 | 9.7 | 86.5 | 0 | 13.5 | 225 | 60 | 99.5 | 0.2 | 0.3 | 99.6 | 99.9 | 99.7 | 0.1 |
| 62a | K12 | 9.7 | 86.5 [g] | 0 | 13.5 | 225 | 60 | 98.9 | 0.6 | 0.5 | 99.2 | 99.6 | 99.5 | 0.2 |

TABLE 6
Decarboxylation of anthranilic acid to aniline without dilution with ANL. Use of the catalyst
as extrudate and reuse thereof.

| | | | AAₜ₌ₜ₀ | ANLₜ₌ₜ₀ | H₂O | T/ | t_{R}/ | ANL_{t=tR} | AA_{t=tR} | AMD_{t=tR} | A_{ANL} | X_{AA} | S_{ANL} | S_{AMD} |
|---|---|---|---|---|---|---|---|---|---|---|---|---|---|---|
| Ex. | Cat. [h] | | [b] | [b] | [b] | ° C. | min | [c] | [c] | [c] | [d] | [e] | [f] | [f] |
| 63 | K12 | 9.7 | 86.5 | 0 | 13.5 | 225 | 60 | 98.3 | 0.5 | 1.2 | 98.6 | 99.6 | 98.9 | 0.5 |
| 64 | K12 | 9.7 | 86.5 | 0 | 13.5 | 225 | 60 | 98.2 | 0.7 | 1.1 | 98.6 | 99.5 | 99.0 | 0.5 |
| 65 | K12 | 9.7 | 86.5 | 0 | 13.5 | 225 | 60 | 98.8 | 0.1 | 1.1 | 98.9 | 99.9 | 99.1 | 0.5 |
| 66 | K12 | 9.7 | 86.5 | 0 | 13.5 | 225 | 60 | 98.7 | 0.4 | 0.9 | 98.9 | 99.7 | 99.2 | 0.4 |
| 67 | K12 | 9.7 | 86.5 | 0 | 13.5 | 225 | 60 | 98.7 | 0.4 | 0.9 | 98.9 | 99.7 | 99.2 | 0.4 |
| 68 | K12 | 9.7 | 86.5 | 0 | 13.5 | 225 | 60 | 99.0 | 0.2 | 0.7 | 99.2 | 99.8 | 99.4 | 0.3 |
| 69 | K12 | 9.7 | 86.5 | 0 | 13.5 | 225 | 60 | 99.0 | 0.1 | 0.9 | 99.1 | 99.9 | 99.2 | 0.4 |
| 70 | K12 | 9.7 | 86.5 | 0 | 13.5 | 225 | 60 | 98.8 | 0.3 | 0.9 | 99.0 | 99.8 | 99.2 | 0.4 |
| 71 | K12 | 9.7 | 86.5 | 0 | 13.5 | 225 | 60 | 98.7 | 0.5 | 0.9 | 98.9 | 99.7 | 99.2 | 0.4 |
| 72 | K12 | 9.7 | 86.5 | 0 | 13.5 | 225 | 60 | 98.9 | 0.3 | 0.8 | 99.1 | 99.8 | 99.3 | 0.3 |
| 73 | K12 | 9.7 | 86.5 | 0 | 13.5 | 225 | 60 | 98.6 | 0.4 | 1.0 | 98.9 | 99.7 | 99.1 | 0.4 |
| 74 | K12 | 9.7 | 86.5 | 0 | 13.5 | 225 | 60 | 99.1 | 0.3 | 0.6 | 99.2 | 99.8 | 99.5 | 0.3 |

### Explanatory Notes for the Tables:

[a] Proportion by mass in % based on the sum total of AA, ANL, H₂O and cat.; catalyst is used as a powder (slurry);
[b] Proportion by mass in % in the reactant mixture, based on the total mass of AA, ANL and H₂O;
[c] Proportion by mass in % in the product mixture, based on the total mass of AA, ANL and 2-aminobenzanilide;
[d] Chemical yield of aniline in %;
[e] Chemical conversion of anthranilic acid in %;
[f] Selectivity for aniline or 2-aminobenzanilide in %;
[g] Fermentatively prepared anthranilic acid;
[h] Proportion by mass in % based on the sum total of AA, ANL, H₂O and cat.; catalyst is used as an extrudate. In each of Examples 63 to 74, the catalyst from the preceding example was reused. Example 74 therefore represents the twelfth use cycle in the context of catalyst recycling over multiple experiments.

As shown by the examples, the process according to the invention enables the conversion of aminobenzoic acid with high conversions and low by-product formation, leading to a high yield of aniline. The catalysts according to the invention enable the formation of aniline in very high yields with a variable composition of the ANL/AA substrate mixture, the water content and a variable process window in relation to the reaction time and temperature. The technical effect in relation to increased aniline yield with the catalysts according to the invention with respect to the prior art was shown. It is possible to use the catalyst as a slurry or extruded solid. Long-term stability of the catalyst was demonstrated without any noticeable drop in the aniline yield over 12 cycles. The catalysts according to the invention are suitable for decarboxylating petrochemically prepared anthranilic acid and biogenically based anthranilic acid.

1. A process for preparing aniline or an aniline conversion product, comprising:
(I) providing aminobenzoic acid;
(II) decarboxylating the aminobenzoic acid to aniline in the presence of an inorganic heterogeneous metal oxide catalyst containing a proportion by mass of Al₂O₃, based on the total mass of metal oxide, of 40.0% to 100%, and a proportion by mass of Al₂O₃, based on the total mass of the inorganic heterogeneous metal oxide catalyst, of 25% to 100%; and
(III) optionally converting the aniline to an aniline conversion product.

2. The process as claimed in claim 1, in which the inorganic heterogeneous metal oxide catalyst contains MgO in a proportion by mass, based on the total mass of metal oxide, of 1.0% to 60.0%.

3. The process as claimed in claim 1, in which the inorganic heterogeneous metal oxide catalyst contains SiO₂ in a proportion by mass, based on the total mass of the inorganic heterogeneous metal oxide catalyst of 1.0% to 30.0%.

4. The process as claimed in claim 1, in which the Al₂O₃ comprises γ-Al₂O₃ or η-Al₂O₃.

5. The process as claimed in claim 1, in which the decarboxylation of the aminobenzoic acid is performed at a temperature of 150° C. to 300° C.

6. The process as claimed in claim 5, in which the decarboxylation of the aminobenzoic acid is performed at an absolute pressure of 0.05 bar to 300 bar.

7. The process as claimed in claim 1, in which the decarboxylation of the aminobenzoic acid is performed in the presence of aniline.

8. The process as claimed in claim 7, in which the decarboxylation of the aminobenzoic acid is performed batchwise and a proportion by mass of aniline, based on the total mass of aniline and aminobenzoic acid, of 0.1% to 90% is established before the start of the decarboxylation.

9. The process as claimed in claim 7, in which the decarboxylation of the aminobenzoic acid is performed continuously and a proportion by mass of aniline, based on the total mass of aniline and aminobenzoic acid, of 0.1% to 90% is always established during the decarboxylation.

10. The process as claimed in claim 1, in which the decarboxylation of the aminobenzoic acid is performed
in the liquid or gas phase in a reactor with an integrated fixed bed of the inorganic heterogeneous metal oxide catalyst,
in the liquid or gas phase in a fluidized bed reactor or
in the liquid phase in a stirred tank with a suspension of the inorganic heterogeneous metal oxide catalyst contained therein.

11. The process as claimed in claim 10, in which the decarboxylation of the aminobenzoic acid is performed in the liquid or gas phase in a reactor with an integrated fixed bed of the inorganic heterogeneous metal oxide catalyst containing a bed of the catalyst as shaped bodies or a configuration of the catalyst as a monolithic structure, the inorganic heterogeneous metal oxide catalyst being regenerated and reused after decarboxylation.

12. The process as claimed in claim 1, in which step (I) comprises fermenting a raw material containing a fermentable carbon-containing compound and a nitrogen-containing compound in the presence of microorganisms.

13. The process as claimed in claim 12, in which the microorganisms comprise Escherichia coli, Pseudomonas putida, Corynebacterium glutamicum, Ashbya gossypii, Pichia pastoris, Hansenula polymorpha, Yarrowia lipolytica, Zygosaccharomyces bailii or Saccharomyces cerevisiae.

14. The process as claimed in claim 1, in which step (III) is performed and comprises:
(1) acid-catalyzed reaction of the aniline with formaldehyde to form di- and polyamines of the diphenylmethane series;
(2) acid-catalyzed reaction of the aniline with formaldehyde to form di- and polyamines of the diphenylmethane series, followed by reaction thereof with phosgene to form di- and polyisocyanates of the diphenylmethane series; or
(3) conversion of the aniline to an azo compound.

15. The process as claimed in claim 1, in which the aminobenzoic acid provided in step (A) comprises ortho-aminobenzoic acid.
